(19) United States
(12) Plant Patent Application Publication
(10) Pub. No.: US 2026/0363154 P1
(43) Pub. Date: Nov. 12, 2026

(54) Strawberry Plant Named 'Sonette'

(50) ;

(71) Applicant: Flevo Berry Holding B.V., Ens (NL)

(72) Inventor: Marcel SUIKER, Ens (NL)

(73) Assignee: Flevo Berry Holding B.V., Ens (NL)

(21) Appl. No.: 19/536,013

(22) Filed: Feb. 10, 2026

(30) Foreign Application Priority Data
May 6, 2025 (QZ) 2025/0813

(51) Int. Cl. A01H 6/74; A01H 5/08

(52) U.S. Cl. PLT/208

(57) ABSTRACT

A new and distinct strawberry plant named ‘Sonette’ particularly distinguished by a compact, upright to semi-upright and uniform plant habit and dense foliage, bright, glossy and uniformly conical shaped fruit, and reduced stolon prominence.

Genus and species: Fragaria x ananassa.

Variety denomination: ‘Sonette’.

### STATEMENT REGARDING PRIOR DISCLOSURES BY THE INVENTOR

The Applicant asserts that no publications or advertisements relating to sales, offers for sale, or public distribution occurred more than one year prior to the effective filing date of this application. Any information about the claimed plant would have been obtained from a direct or indirect disclosure from the Inventor. The Applicant claims a prior art exemption under 35 U.S.C. 102(b)(1) for disclosures and/or sales that fall within a one-year grace period prior to the filing date. This application claims priority to CPVO application no. 2025/0813 filed on May 6, 2025, which is herein incorporated by reference.

### BACKGROUND OF THE NEW PLANT

The present invention comprises a new and distinct variety of strawberry plant, botanically known as Fragaria x ananassa, and hereinafter referred to by the variety name ‘Sonette’. The new strawberry variety ‘Sonette’ is a seedling selection that resulted from a controlled pollination breeding program carried out in April of 2018 in Ens, Netherlands. The new variety originated as a seedling from crossing the proprietary strawberry breeding line ‘08-073’ (unpatented), as the seed parent, with the proprietary strawberry breeding line ‘11-003’ (unpatented) as the pollen parent. Seed from said cross was harvested, then germinated, and the resulting seedlings were grown to a mature size in order to evaluate for desirable commercial characteristics. In August of 2019, the inventor selected the claimed strawberry variety from amongst said seedlings for further evaluation to determine its distinctness and stability of the unique characteristics first observed.

Asexual reproduction of ‘Sonette’, by way of vegetative cuttings, was first performed in September of 2019 in Ens, Netherlands. Through seven subsequent generations of asexual propagation, the unique features of this new strawberry variety ‘Sonette’ have proven to be stable and remain true to type.

### SUMMARY OF THE INVENTION

The new strawberry variety ‘Sonette’ has not been observed under all possible environmental conditions. The phenotype may vary somewhat with variations in environment such as temperature, day length, light intensity, water status, fertilizer rate and type, without, however, any variance in genotype.

The following are the most outstanding and distinguishing characteristics of this new strawberry variety. The combination of these characteristics distinguishes ‘Sonette’ as a new and distinct variety of strawberry.
1. Strawberry ‘Sonette’ exhibits a compact, upright to semi-upright and uniform plant habit and dense foliage;
2. Strawberry ‘Sonette’ exhibits bright, glossy and uniformly conical shaped fruit; and
3. Strawberry ‘Sonette’ exhibits reduced stolon prominence.

### DESCRIPTION OF THE PHOTOGRAPHS

This new strawberry variety is illustrated by the accompanying-colored photographs which show the overall appearance and distinct characteristics of the plant. The colors shown are as true as can be reasonably obtained by conventional photographic procedures. The photographs are of 12-month-old plants grown in 20-centimeter square containers in a conventional cold glasshouse in Ens, Netherlands. Colors in the photographs may differ slightly from the color values cited in the botanical description which accurately describes the colors of the new variety.

FIG. 1. shows the overall plant form, foliage, and fruit of ‘Sonette’.

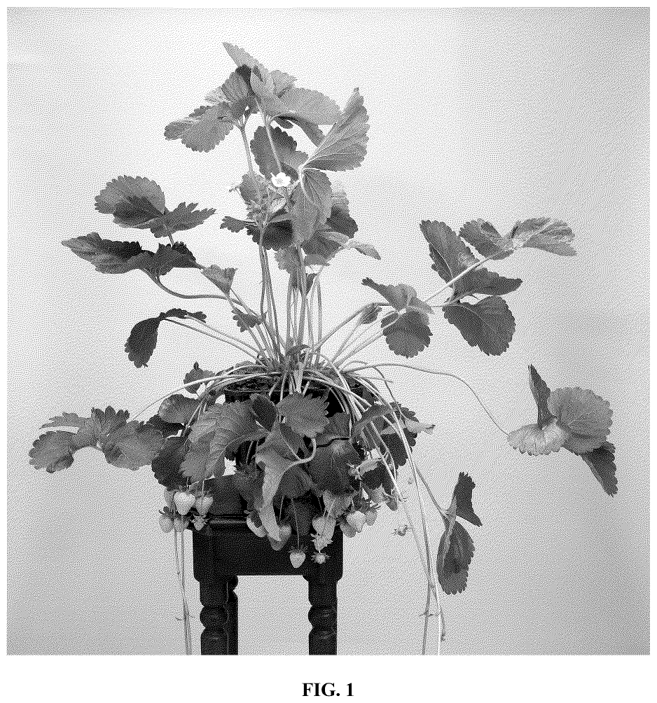

FIG. 2. shows a close-up of a typical leaf observed on plants of ‘Sonette’.

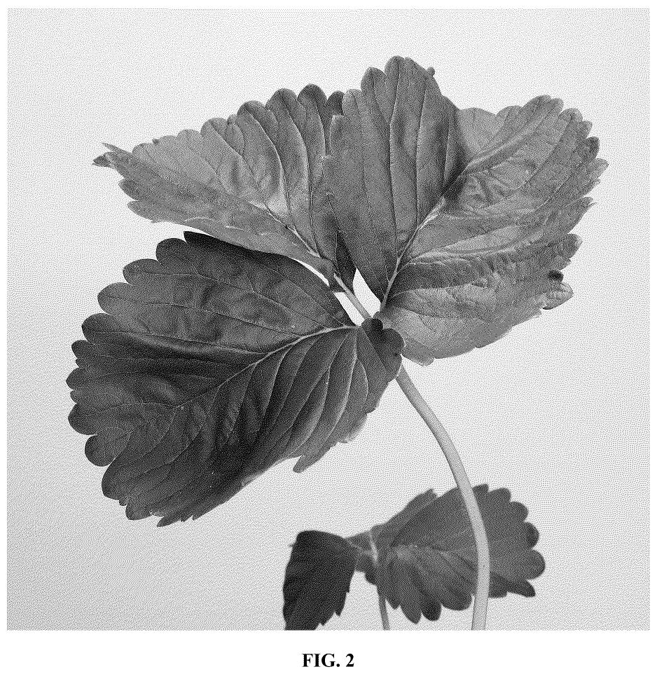

FIG. 3. shows a close-up of a typical flower observed on plants of ‘Sonette’.

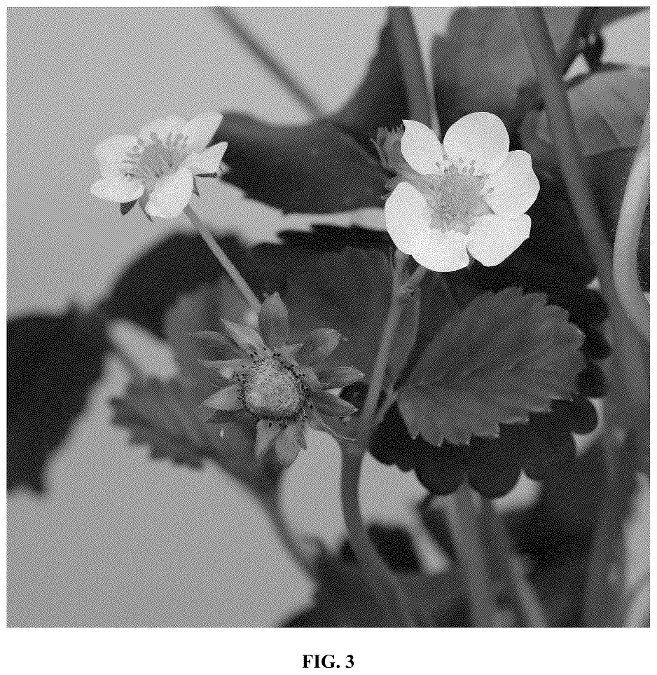

FIG. 4. shows typical fruit observed on plants of ‘Sonette’.

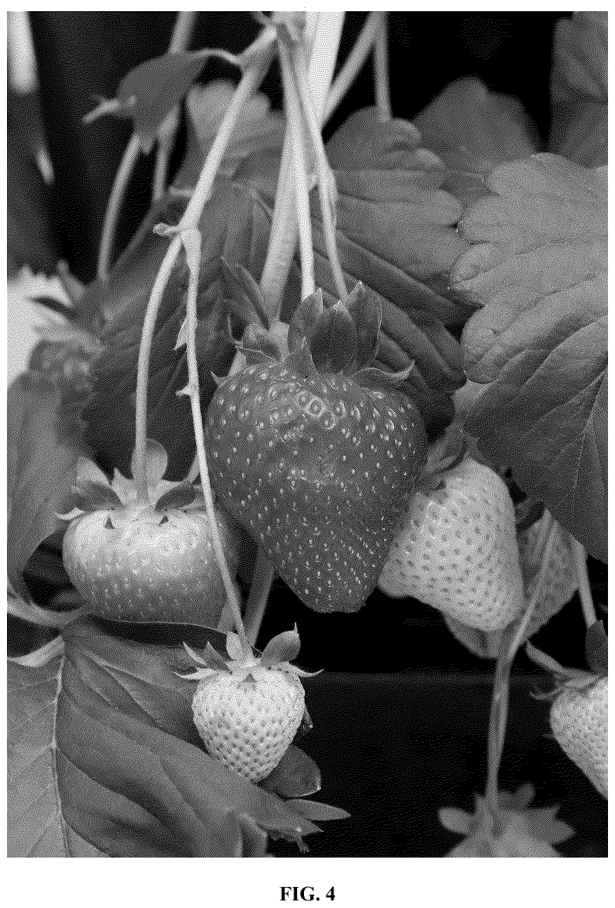

### DESCRIPTION OF THE NEW VARIETY

The following is a detailed botanical description of the new and distinct variety of strawberry known as ‘Sonette’, based upon observations of 12-month-old plants grown in 29-centimeter square containers in a conventional cold greenhouse located in Ens, Netherlands. Plants were grown on benches in a non-climate-controlled greenhouse, using conventional production protocols for strawberry plants which consisted of drip irrigation with fertigation applications as needed. Biological insect control measures were utilized in production. Plants were produced with full sun exposure and no photoperiodic treatments or artificial light was given to the plants. Temperatures ranged from 18 to 30 degrees Celsius during the day and 5 to 18 degrees Celsius at night. Observation data was recorded in June of 2025. Those skilled in the art will appreciate that certain characteristics will vary with older or, conversely, with younger plants. ‘Sonette’ has not been observed under all possible environmental conditions. Where dimensions, sizes, colors and other characteristics are given, it is to be understood that such characteristics are approximations or averages set forth as accurately as practicable. The phenotype of the new variety may differ from the descriptions set forth herein with variations in environmental, climatic and cultural conditions. In the following description, color references are made to The Royal Horticultural Society Colour Chart, Sixth Edition 2019 reprint, except where general color terms of ordinary dictionary significance are used.

### DETAILED BOTANICAL DESCRIPTION

Classification:
   Family.—Rosaceae.
   Botanical.—Fragaria x ananassa.
   Denomination.—‘Sonette’.
   Common name.—Strawberry.
Plant description:
   Growth habit.—Acaulescent, stoloniferous herbaceous perennial with leaves and inflorescence developing from basal rosettes with approximately 5 rosettes per plant; no lateral branching is present.
   Overall plant shape.—Rounded with pendulous stolons.
   Average height.—43.0 cm to the top of the foliar plane; 27.0 cm to the top of the floral plane.
   Plant diameter or spread.—Average 72.8 cm.
   Plant vigor.—Moderate to high.
   Growth rate.—Moderate.
Stolon description:
   Appearance and shape.—Pendulous (or creeping when not grown in hanging baskets); circular in cross section.
   Aspect (angle from vertical).—Average 70 degrees, but curled downward to vertical.
   Length.—70.2 cm.
   Diameter.—0.3 cm.
   Internode length.—16.8 cm.
   Strength.—Strong.
   Texture.—Sparsely to moderately covered with very thin, short hairs with an average length of 0.1 cm; hairs are colored a transparent white but too sparsely present to measure RHS color designation.
   Luster.—Matte.
   Color.—Immature stolons: Near N144C (yellow-green). Mature stolons: Near 144B (yellow-green). At internodes: Near 144C (yellow-green).
Foliage description:
   Arrangement.—Alternate in basal rosettes.
   Attachment.—Petiolate.
   Division.—Trifoliate.
   Compound leaf outline.—Reniform.
   Length.—16.6 cm, excluding the petiole.
   Width.—19.8 cm.
   Quantity.—6 leaves per rosette.
   Leaflets.—Quantity: 3, and occasionally 4 or 5. Shape: Varying from broad obovate to broad ovate, occasionally near orbicular. Length: 10.8 cm. Width: 10.3 cm. Aspect: Strongly concave. Apex: Broadly acute. Base: Terminal leaflet: Broadly acuminate. Lateral leaflets: Unequally obtuse. Margin: Coarsely dentate to crenate. Texture: Adaxial surface: Smooth and sparsely to moderately covered with very thin, soft hairs with an average length of 0.1 cm; hairs are colored a transparent greyed-white but too small and too sparsely present to accurately measure RHS color designation. Abaxial surface: Smooth and sparsely to moderately covered with very thin, soft adpressed hairs with an average length of 0.1 cm; hairs are colored a transparent white but too small and too sparsely present to measure RHS color designation. Rugosity (adaxial and abaxial surfaces): Slightly rugose. Luster: Adaxial surface: Very slightly glossy. Abaxial surface: Matte. Color: Juvenile foliage: Adaxial surface: Near 144A (yellow-green) with dentate to crenate marginal tips colored near a blend of 139A and 141A (green). Abaxial surface: Near 144B (yellow-green) with veins colored near 145C (yellow-green). Mature foliage: Adaxial surface: Near 137B (green). Abaxial surface: 147D (green). Venation: Type: Pinnate. Color (mature leaf): Adaxial surface: Near N144C (yellow-green). Abaxial surface: Near 145C (yellow-green).
   Petiole.—Profile: Flattened on the upper side and occasionally slightly triangular in cross section. Length: 29.6 cm. Diameter: Average 0.5 cm horizontally and average 0.4 cm vertically. Strength: Strong. Texture: Moderately to strongly covered with very thin, soft hairs with an average length of 0.2 cm; hairs are colored a transparent white but too small and too sparsely present to measure RHS color designation. Luster (adaxial and abaxial surfaces): Moderately glossy. Color (adaxial and abaxial surfaces): Near 144B (yellow-green).
   Stipules.—General: Two leafy stipules present at the base of each petiole. Shape: Lanceolate. Length: 2.8 cm. Width: 0.5 cm. Apex: Narrow acute. Base: Broad cuneate. Color (adaxial and abaxial surfaces): Near 184B (greyed-purple) and primary vein colored 145C (yellow-green). Texture: Adaxial surface: Glabrous. Abaxial surface: Moderately covered with very thin, soft hairs with an average length of 0.1 cm; hairs are colored a transparent white but too small and too sparsely present to measure RHS color designation.
Inflorescence description:
   Type.—Compound cymose panicle.
   Natural flowering season.—From spring to summer in Ens, Netherlands.
   Time to flower.—9 months.
   Height.—Average 8.9 cm, excluding the peduncle.
   Diameter.—Average 7.9 cm.
   Quantity of flowers per inflorescence.—5.
   Quantity of flower buds per plant.—10.
   Quantity of flowers per plant.—50.
   Quantity of flowers and flower buds per plant.—50.
   Peduncle.—Length: 24.4 cm. Diameter: 0.4 cm. Aspect: Average of 50 degrees to horizontal (varying between 30 degrees to 75 degrees). Strength: Moderate to strong. Texture: Moderately to densely covered with thin, soft hairs with an average length of 0.2 cm; hairs are colored a transparent near NN155D (white). Luster: Slightly glossy. Color: 144C (yellow-green).
Flower buds:
   Shape.—Ovate to circular.
   Length.—1.1 cm.
   Diameter.—2.4 cm.
   Texture.—Smooth and glabrous except for the immature sepals which are moderately covered with very thin, soft hairs with an average length of 0.75 mm; hairs are colored a transparent white but too small and too sparsely present to measure RHS color designation.
   Luster.—Matte.
   Color.—Sepals colored a blend of near 139B and 143A (green) and fading at the base to near 144B (green); immature petals colored near 155C (white) and fading at the base to near 3C (yellow).
Flower:
   Form.—Rotate; single.
   Aspect.—Upright to outwardly.
   Vertical height.—3.1 cm.
   Horizontal diameter.—3.1 cm.
   Depth.—1.2 cm.
   Fragrance.—Faint, sweet and fruity.
   Lastingness.—Approximately 7 days.
   Persistence.—Self-cleaning.
   Pedicels.—Length: 4.8 cm. Diameter: 0.2 cm. Aspect: Approximately 17.5 degrees to the peduncle axis (varying between 0 degrees and 30 degrees). Strength: Moderate. Texture: Moderately covered with thin, soft hairs with an average length of 0.5 mm; hairs are colored a transparent near NN155D (white). Luster: Slightly glossy. Color: Near 144D (yellow-green).
   Petals.—Arrangement: Rotate with a single whorl of free petals. Quantity: 5 or 6. Shape: Orbicular and very slightly concave. Length: 1.2 cm. Width: 1.2 cm. Apex: Obtuse. Base: Broad cuneate. Margin: Entire, slightly undulated. Texture (adaxial and abaxial surfaces): Glabrous, smooth and moderately velvety. Rugosity: Non-rugose. Luster (adaxial and abaxial surfaces): Matte. Color when opening: Adaxial surface: NN155C (white) and fading at the base to near NN155A (white). Abaxial surface: NN155C (white) and fading at the base to near NN155A (white). Color when fully opened: Adaxial surface: NN155C (white) and fading at the base to near 2C (yellow). Abaxial surface: Near NN155 and fading at the base to near 3D (yellow). Color when fading (adaxial and abaxial surfaces): Petals do not fade with age. Venation color when fully opened (adaxial and abaxial surfaces): Not visible.
   Calyx.—Shape: Rotate. Length: 0.7 cm. Diameter: 1.9 cm. Sepals: Arrangement: Rotate and fused at the base. Quantity: Average 12. Length: 1.1 cm. Width: 0.3 cm. Shape: Elliptic to obovate. Apex: Acute. Base: Cuneate, fused. Margin: Entire. Texture: Adaxial surface: Smooth, sparsely covered with very thin, soft hairs with an average length of 0.5 mm; hairs are colored a transparent white but too small and too sparsely present to measure RHS color designation. Abaxial surface: Smooth, sparsely covered with very thin, soft hairs with an average length of 0.5 mm; hairs are colored a transparent white, but too small and too sparsely present to accurately measure RHS color designation. Luster (adaxial and abaxial surfaces): Matte. Color when opening: Adaxial surface: Near a blend of 139B and 143A (green) and fading at the base to near 144B (yellow-green). Abaxial surface: Near 143C (green). Color when fully opened: Adaxial surface: Upper ring: Near 143C (green) with darker colored apices near 143B (green) and bases N144B (yellow-green). Lower ring: Near a blend of 137B and 137C (green). Abaxial surface: Upper ring: Near 143C (green) with bases near 144B (yellow-green). Lower ring: Near 143B (green). Sepal spurs: Not present.
   Bracts.—Position: One bract present at the base of each pedicel. Shape: Ovate to orbicular. Length: Average 4.4 cm. Width: Average 4.0 cm. Apex: Rounded. Base: Obtuse. Margins: Crenate to dentate. Color: Adaxial surface: Near a blend of 137C and 143A (green). Abaxial surface: Near 147C (yellow-green). Texture: Adaxial surface: Very sparsely covered with thin, soft hairs with an average length of 0.5 mm; hairs are colored a transparent white but too small and too sparsely present to measure RHS color designation. Abaxial surface: Sparsely covered with thin, soft hairs with an average length of 0.1 cm; hairs are colored a transparent white but too small and too sparsely present to measure RHS color designation.
   Stipules.—Position: Two stipules present at each node of the inflorescence. Shape: Elliptic to ovate and slightly carinate. Length: Average 1.2 cm. Width: 0.5 cm. Apex: Acute. Base: Cuneate. Color: Adaxial surface: Near 144A (yellow-green) and occasionally edged 184C (greyed-purple). Abaxial surface: Near 144A (yellow-green) and occasionally edged 184D (greyed-purple). Texture: Adaxial surface: Very sparsely covered with thin, soft hairs with an average length of 0.5 mm; hairs are colored a transparent white but too small and too sparsely present to measure RHS color designation. Abaxial surface: Sparsely covered with thin, soft hairs with an average length of 0.1 cm; hairs are colored a transparent white but too small and too sparsely present to measure RHS color designation.
Reproductive organs:
   Stamens.—Quantity per flower: Average 27. Anthers: Shape: Broad elliptic to oblong. Length: 0.1 cm. Width: 0.1 cm. Color: 17B (yellow-orange). Filaments: Length: 0.3 cm. Color: Near 151D (yellow-green), distal end lighter near 155A (yellow-green). Pollen: Abundance: Low. Color: Near 16A (yellow-green).
   Pistil.—Quantity per flower: 240. Length: 0.15 cm. Style: Length: 1.2 mm. Color: Near 150C (yellow-green). Stigma: Shape: Club-shaped. Length: 0.25 mm. Diameter: 0.25 mm. Color: Near 8B (yellow).
   Ovary color.—Near 150C (yellow-green).
Seed and fruit:
   Fruit.—Harvest season: June in the Netherlands. Time to maturity: Approximately 5 weeks after flowering. Quantity per plant: 30. Dimensions: 4.8 cm long and 4.8 cm in diameter. Weight: 14.8 grams. Shape: Conical. Texture: Smooth and glabrous. Luster: Glossy. Mature fruit surface color: Near 44A (red). Mature fruit flesh color: Dark red. Immature fruit surface color: Near 145C to 145D (light yellow green). Immature fruit flesh color: Near 157D (greenish white). Fruit flavor: Sweet and pleasantly acidic. Firmness of flesh: Soft. Skin: Firm. Lastingness of fruit: Approximately 1 week. Yield: Approximately 6.0 to 6.5 kilograms of Class 1 fruit per linear meter of planting; however, yield is highly dependent on the cultivation system, such as planting density. Storability: Good. Market use: Fresh fruit market.
   Achenes.—Position: Level with fruit surface. Quantity per fruit: 200. Length: 0.1 cm. Diameter 0.8 mm. Texture: Smooth and glabrous. Color: 144A (yellow-green).
Environmental tolerances: Adapted to USDA Zones 4 through 9 and temperatures ranging from at least minus 32 degrees Celsius to 40 degrees Celsius; low to moderate tolerance to rain; moderate to high tolerance to wind.
Disease and pest tolerance: No claims are made to any superior disease or insect resistance or tolerance.

### COMPARISON WITH THE PARENT PLANTS

The new strawberry variety ‘Sonette’ can be distinguished from its seed parent ‘08-073’ (unpatented) in the following characteristics described in Table 1 below.

TABLE 1

| Characteristic | ‘Sonette’ | ‘08-073’ |
|---|---|---|
| Plant habit. | More compact, upright | More spreading with |
| | to semi-upright and uniform | looser overall foliage |
| | with dense foliage. | structure. |
| Stolon | Less prominent and shorter. | Stolons long and clearly |
| development. | | trailing. |
| Fruit position. | Held closer to the pot and | Fruit hangs lower on |
| | partially supported by the | longer peduncles. |
| | foliage. | |
| Fruit shape | Uniformly conical with a | Short conical to more |
| uniformity. | well-defined apex. | rounded in shape. |
| Fruit surface | Smooth and glossy with | Less glossy |
| appearance. | evenly distributed achenes. | with irregular |
| | | achene distribution. |

The new strawberry variety ‘Sonette’ can be distinguished from its pollen parent ‘11-003’ (unpatented) in the following characteristics described in Table 2 below.

TABLE 2

| Characteristic | ‘Sonette’ | ‘11-003’ |
|---|---|---|
| Plant habit. | More compact, upright | More spreading, |
| | to semi-upright and uniform | elongated and trailing. |
| | with dense foliage. | |
| Stolon | Less prominent and shorter. | Long, thin and highly |
| Development. | | trailing. |
| Leaf | Leaves held closer together | More widely spaced and |
| arrangement. | forming a dense canopy. | outwardly spreading. |
| Fruit position. | Held closer to the crown and | Borne on long |
| | partially supported by the | peduncles and hanging |
| | foliage. | well below the container. |
| Fruit shape | Uniformly conical with | Variable, including short |
| uniformity. | a well-defined apex. | conical to irregular |
| | | shapes. |

### COMPARISON WITH RELATED KNOWN VARIETY

The new strawberry variety ‘Sonette’ can be distinguished from the related known variety of strawberry plant ‘Florice’ (U.S. Plant Pat. No. 34,684) in the following characteristics described in Table 3 below.

TABLE 3

| Characteristic | ‘Sonette’ | ‘Florice’ |
|---|---|---|
| Plant habit. | More compact, upright | More spreading with |
| | to semi-upright and uniform | longer, trailing growth. |
| | with dense foliage. | |
| Stolon | Less prominent and shorter. | Stolons long and clearly |
| development. | | trailing. |
| Fruit shape | Uniformly conical with a | Short conic, broader, and |
| uniformity. | well-defined apex. | less uniform in shape. |
| Mature fruit | Bright, glossy red. | Red, less glossy. |
| external color. | | |
| Fruit surface | Smooth and glossy with | Slightly uneven surface |
| appearance. | evenlydistributed achenes. | with irregular achene |
| | | distribution. |

What is claimed is:

1. A new and distinct variety of strawberry plant named ‘Sonette’, substantially as illustrated and described herein.
